(19) United States
(12) Patent Application Publication
(10) Pub. No.: US 2008/0319307 A1
(43) Pub. Date: Dec. 25, 2008

(54) Method for medical imaging using fluorescent nanoparticles

(75) Inventors: James W. Voegele, Cincinnati, OH (US); Robert P. Gill, Mason, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc.

(21) Appl. No.: 11/820,354

(22) Filed: Jun. 19, 2007

(51) Int. Cl. A61B 5/055

(52) U.S. Cl. 600/414; 977/930

(57) ABSTRACT

A first method for medically imaging a patient includes obtaining a carrier medium containing fluorescent nanoparticles. The carrier medium containing the fluorescent nanoparticles is disposed inside the patient at a site where the carrier medium will migrate to specific tissue of known shape. The nanoparticles are activated to fluoresce. Image data of the patient is obtained including the specific tissue after the specific tissue has picked-up the fluorescing fluorescent nanoparticles. The known shape of the specific tissue is identified in the image data. An image representation of the image data is created using the identified known shape as a fiducial. An image of the image representation of the image data is displayed. In a second method, the carrier medium is injected inside tissue of the patient at a site where the carrier medium will be localized in the injected tissue, and the injecting deposits the medium in a predetermined shape in the tissue.

### FIELD OF THE INVENTION

The present invention is related generally to medical images, and more particularly to a method for medical imaging using fluorescent nanoparticles.

### BACKGROUND OF THE INVENTION

Imagers are known for obtaining image data of a patient and for displaying images of the image data on a display monitor. Such images include, without limitation, ultrasound images, X-ray images, computerized tomography (CT) images, positive electron emission (PET) images, magnetic resonance (MRI) images, fluoroscope images, etc. Where needed, it is known to register these images with a real world object by placing a fiducial component on the skin of the patient, wherein the fiducial component has a predetermined shape, and wherein the fiducial component is recognizable as a fiducial in the image data using pattern recognition software (e.g., a conventional segmentation subroutine).

Fluorescent nanoparticles (sometimes called fluorescent photophores) are nano-particles which exhibit fluorescence and include, without limitation, quantum dots and Cornell dots. It is known to use quantum dots for in vivo medical imaging. Examples of quantum dots include Kodak's X-Sight imaging agents. Quantum dots exposed to electromagnetic radiation (such as visible or infra-red light) at a first frequency will then emit electromagnetic radiation light at a second frequency. In one known application, the quantum dots are employed in a carrier medium which is tailored to be picked-up by a target tissue inside the patient. In one experiment, the emitted radiation from quantum dots picked-up by the sentinel lymph node of a mouse one centimeter beneath the skin was detected through the skin by a fluorescence imaging system located outside the mouse. Encapsulation of quantum dots enabling the quantum dots to function over an extended period of time is described in US Patent Application Publication No. 2004/0105979. Cornell dots (fluorescent silica-based nanoparticles) are available from Hybrid Silica Technologies, Inc. of 105 White Park Road, Ithaca, N.Y. 14850 and are described in US Patent Application Publication Nos. 2004/0101822, 2006/0183246 and 2006/0245971.

Still, scientists and engineers continue to seek improvements in medical imaging.

### SUMMARY

A first method of the invention is for medically imaging a patient and includes several steps. One step includes obtaining a carrier medium containing fluorescent nanoparticles. Another step includes disposing the carrier medium containing the fluorescent nanoparticles inside the patient at a site where the carrier medium will migrate to, and be picked up by, specific tissue of the patient, wherein the specific tissue has a known shape. Another step includes activating the fluorescent nanoparticles to fluoresce. Another step includes obtaining image data of the patient including the specific tissue after the specific tissue has picked-up the fluorescing fluorescent nanoparticles. Another step includes identifying the known shape of the specific tissue in the image data. Another step includes creating an image representation of the image data using the identified known shape as a fiducial. Another step includes displaying an image of the image representation of the image data.

A second method of the invention is for medically imaging a patient and includes several steps. One step includes obtaining a carrier medium containing fluorescent nanoparticles. Another step includes injecting the carrier medium containing the fluorescent nanoparticles inside tissue of the patient at a site where the carrier medium will be localized in the injected tissue, wherein the injecting deposits the carrier medium in a predetermined shape in the injected tissue. Another step includes activating the fluorescent nanoparticles to fluoresce. Another step includes obtaining image data of the patient including the injected tissue. Another step includes identifying the predetermined shape of the injected carrier medium in the image data. Another step includes creating an image representation of the image data using the identified predetermined shape as a fiducial. Another step includes displaying an image of the image representation of the image data.

Several benefits and advantages are obtained from one or more of the methods of the invention. In one example of the first method, the fluorescent nanoparticles in the image data act as a fiducial in creating an image representation of the image data, wherein the known shape of the specific tissue is more readily identified by the computer as the fiducial when fluorescent nanoparticles are used than when fluorescent nanoparticles are not used. In one example of the second method, the fluorescent nanoparticles in the image data act as a fiducial in creating an image representation of the image data, wherein the fluorescent nanoparticles in the predetermined shape of the injected carrier medium allow identification by the computer of the predetermined shape as the fiducial.

### BRIEF DESCRIPTION OF THE FIGS.

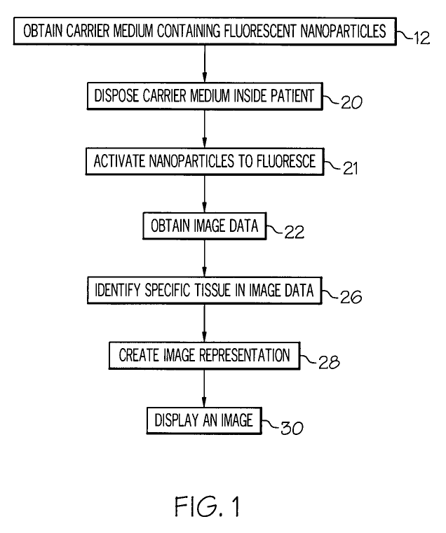

FIG. 1 is a flow chart of a first method of the invention;

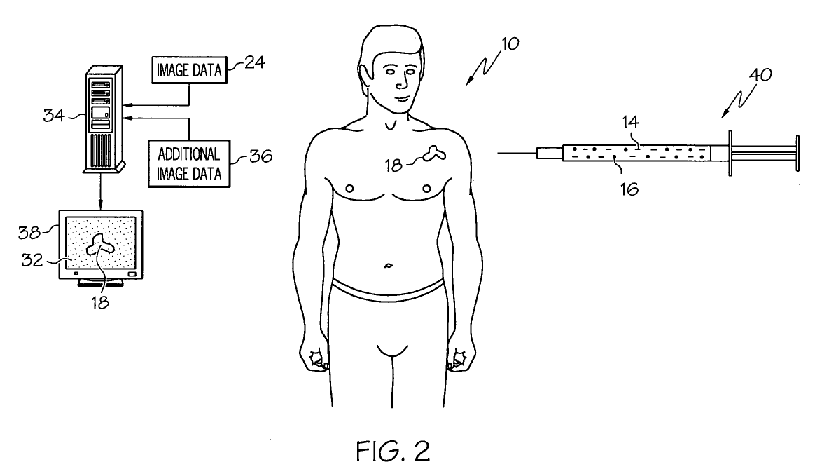

FIG. 2 is a is an explanatory diagram illustrating one embodiment for carrying out the method of FIG. 1;

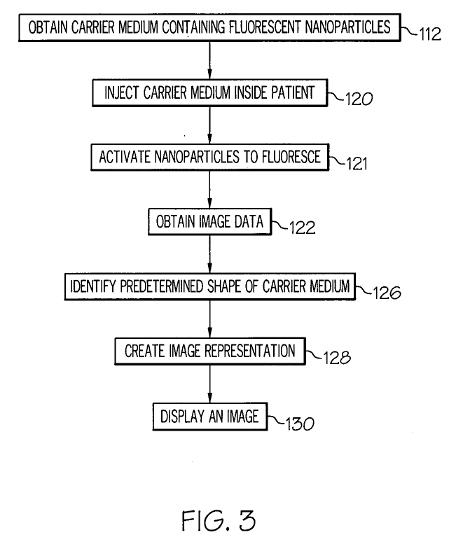

FIG. 3 is a flow chart of a second method of the invention; and

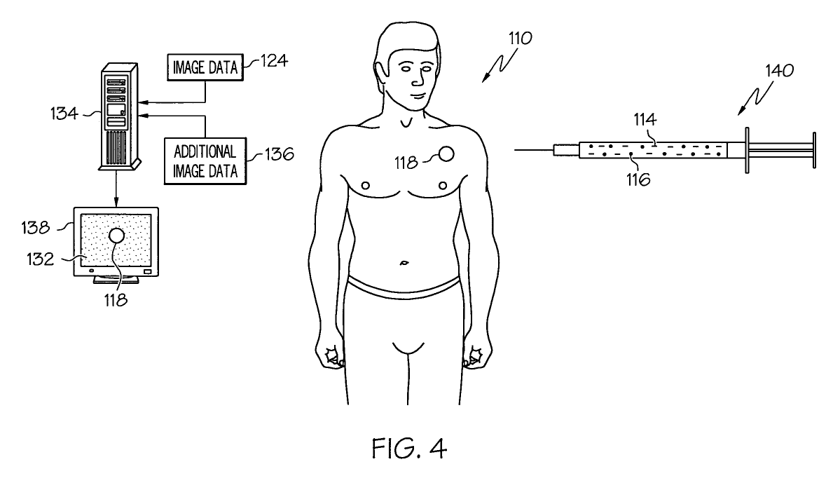

FIG. 4 is a is an explanatory diagram illustrating one embodiment for carrying out the method of FIG. 3.

### DETAILED DESCRIPTION

Before explaining the several methods of the present invention in detail, it should be noted that the present invention is not limited in its application or use to the details of construction and arrangement of parts and steps illustrated in the accompanying drawings and description. The illustrative methods of the invention may be implemented or incorporated in other embodiments, methods, variations and modifications, and may be practiced or carried out in various ways. Furthermore, unless otherwise indicated, the terms and expressions employed herein have been chosen for the purpose of describing the illustrative methods of the present invention for the convenience of the reader and are not for the purpose of limiting the invention.

It is further understood that any one or more of the following-described implementations, examples etc. can be combined with any one or more of the other following-described implementations, examples etc.

A first method of the invention is shown in FIGS. 1-2, is for medically imaging a patient 10, and includes steps a) through g). Step a) is labeled as “Obtain Carrier Medium Containing Fluorescent Nanoparticles” in block 12 of FIG. 1. Step a) includes obtaining a carrier medium 14 containing fluorescent nanoparticles 16. Step b) is labeled as “Dispose Carrier Medium Inside Patient” in block 20 of FIG. 1. Step b) includes disposing the carrier medium 14 containing the fluorescent nanoparticles 16 inside the patient 10 at a site where the carrier medium 14 will migrate to, and be picked up by, specific tissue 18 of the patient 10, wherein the specific tissue 18 has a known (two-dimensional or three-dimensional) shape. Step c) is labeled as “Activate Nanoparticles To Fluoresce” in block 21 of FIG. 1. Step c) includes activating the fluorescent nanoparticles 16 to fluoresce. Step d) is labeled as “Obtain Image Data” in block 22 of FIG. 1. Step d) includes obtaining image data 24 of the patient 10 including the specific tissue 18 after the specific tissue 18 has picked-up the fluorescing fluorescent nanoparticles 16. Step e) is labeled as “Identify Specific Tissue In Image Data” in block 26 of FIG. 1. Step e) includes identifying the known shape of the specific tissue 18 in the image data 24. Step f) is labeled as “Create Image Representation” in block 28 of FIG. 1. Step f) includes creating an image representation of the image data 24 using the identified known shape as a fiducial. Step g) is labeled as “Display An Image” in block 30 of FIG. 1. Step g) includes displaying an image 32 of the image representation of the image data 24.

It is noted that the carrier medium 14, including the fluorescent nanoparticles 16, will find its way to the specific tissue 18, will be collected within the specific tissue 18, and will be held within the specific tissue 18 for a period of time. An example of such a carrier medium 14 is water, an example of a site is a tumor, and an example of a specific tissue 18 is a sentinel lymph node, wherein the water will migrate from the tumor to the sentinel lymph node via the lymphatic system of the patient 10. It is also noted that eventually the fluorescent nanoparticles 16 will stop fluorescing and the carrier medium 14, including the fluorescent nanoparticles 16, will leave the specific tissue 18.

In a first implementation of the first method, the image data 24 is magnetic resonance (MRI) image data. In a second implementation, the image data 24 is computerized tomography (CT) image data. In a third implementation, the image data 24 is positive electron emission (PET) images. In a fourth implementation, the image data 24 is fluoroscope image data. Other implementations are left to those skilled in the art.

In one enablement of the first method, steps e) and f) are performed by a computer 34. In one variation, step e) is performed by the computer 34 using a segmentation subroutine. In one example, the displayed image 32 is of real-time (i.e., substantially real-time noting computer processing delays) image data. In another example, the displayed image is of pre-acquired image data.

In one employment of the first method, the specific tissue 18 is tissue which is to be medically treated. In one example, the medical treatment is performed using the displayed image 32, wherein the displayed image 32 of real-time image data. In one variation, the specific tissue 18 is diseased tissue. Other examples are left to the artisan.

In an extension of the first method, there is also included steps h) through j). Step h) includes obtaining additional image data 36 of the patient 10 including the specific tissue 18. Step i) includes creating an image representation of the additional image data 36. Step j) includes displaying an image (which would be superimposed on image 32 in FIG. 2) of the image representation of the additional image data 36 registered and overlaid with the displayed image 32 of the image representation of the image data 24. In one variation, which is especially useful when one of the images is a real-time image, additional conventional software is used to synchronize the images to account for tissue movement (such as that caused by respiration or peristalsis).

In one variation of the extension of the first method, the additional image data 36 and the image data 24 are obtained from different imaging modalities (e.g., an MRI imager and a CT imager, etc.) In one implementation, the image data 32 is pre-acquired image data and the additional image data 36 is real-time image data. In one example, without limitation, a person who has had MRI images taken with the fluorescent nanoparticles 16 in an imaging area of a medical facility where steps a) through d) were performed is then quickly moved to a surgical area of the medical facility where steps e) through j) are performed, wherein step h) obtains ultrasound image data and wherein surgery is then carried out using the overlaid images.

In one enablement of the extension of the first method, the additional image data 36 and the image data 24 are obtained from using the physically same fluorescent nanoparticles or from using physically different fluorescent nanoparticles. In one example, the image representation of the image data 24 and the image representation of the additional image data 36 are registered to a reference coordinate system (allowing for image overlaying), wherein the reference coordinate system is defined by using the known shape of the specific tissue 18. It is noted that code can be written by those of ordinary skill in the art, without undue experimentation, which instructs the computer 34 to create the image representation of the image data 24 registered to the reference coordinate system and to create the image representation of the additional image data 36 registered to the reference coordinate system.

In a different enablement of the extension of the first method, the additional image data 36 is obtained without using any fluorescent nanoparticles. In one example, a position sensor (not shown) is placed on or in the patient 10 at a known location and orientation with respect to a manufactured fiducial placed on or in the patient and recognized in the additional image data 36 by the computer 34. In another example, the position sensor is adapted to also act as the manufactured fiducial. This allows the additional image data 36 to be registered to a reference coordinate system. In this example, the image data 24 can also be registered to the reference coordinate system, when the position sensor is at a known location and orientation with respect to the specific tissue 18, allowing for image overlaying. It is noted that code can be written by those of ordinary skill in the art, without undue experimentation, which instructs the computer 34 to create the image representation of the image data 24 registered to the reference coordinate system and to create the image representation of the additional image data 36 registered to the reference coordinate system.

Examples of position sensors adapted to provide position data include, without limitation, the position sensors of the AC-based position sensing system available from Biosense-Webster and the DC-based position sensing system available from Ascension Technology Corporation. It is noted that the term “position” includes up to six degrees of freedom so that calculating position includes calculating a two-dimensional or three-dimensional location (translation) and two or three degrees of orientation (alignment) of the sensor with respect to a reference coordinate system. A description of the operation of an embodiment of a position sensor adapted to provide position data is found in US Patent Application Publication 2006/0089624.

In one application of the first method, step g) displays the image 32 on a monitor 38. Examples of a display monitor 38 include, without limitation, a computer monitor, a goggle display screen, and a room wall upon which projected images are displayed. In one variation, the image 32 is a manipulative 3D (3 dimensional) image. An example of a computer program which creates a manipulative 3D display image from 2D CT-scans and MRI-scans is Mimics available from Materialise of Ann Arbor, Mich.

In one embodiment of the first method, the carrier medium 14, including the fluorescent nanoparticles 16, is disposed inside the patient 10 using a syringe 40. In a first variation, the distal tip of the syringe 40 pierces the external skin of the patient. In a second variation, not shown, the distal tip of the syringe extends from the distal end of an endoscope which is disposed inside a body lumen or internal surface of the patient 10, wherein the distal tip of the syringe pierces the outer layer of a wall of the body lumen.

A second method of the invention is shown in FIGS. 3-4, is for medically imaging a patient 110, and includes steps a) through g). Step a) is labeled as “Obtain Carrier Medium Containing Fluorescent Nanoparticles” in block 112 of FIG. 3. Step a) includes obtaining a carrier medium 114 containing fluorescent nanoparticles 116. Step b) is labeled as “Inject Carrier Medium Inside Patient” in block 120 of FIG. 3. Step b) includes injecting the carrier medium 114 containing the fluorescent nanoparticles 116 inside tissue 118 of the patient 110 at a site where the carrier medium 114 will be localized in the injected tissue 118, wherein the injecting deposits the carrier medium 114 in a predetermined (two-dimensional or three-dimensional) shape in the injected tissue 118. Step c) is labeled as “Activate Nanoparticles To Fluoresce” in block 121 of FIG. 3. Step c) includes activating the fluorescent nanoparticles 116 to fluoresce. Step d) is labeled as “Obtain Image Data” in block 122 of FIG. 3. Step d) includes obtaining image data 124 of the patient 110 including the injected tissue 118. Step e) is labeled as “Identify Predetermined Shape Of Carrier Medium” in block 126 of FIG. 3. Step e) includes identifying the predetermined shape of the injected carrier medium 114 in the image data 124. Step f) is labeled as “Create Image Representation” in block 128 of FIG. 3. Step f) includes creating an image representation of the image data 24 using the identified predetermined shape as a fiducial. Step g) is labeled as “Display An Image” in block 130 of FIG. 3. Step g) includes displaying an image 132 of the image representation of the image data 124.

It is noted that the carrier medium 114 will maintain its position and shape in the injected patient tissue 118 for a period of time. An example of such a carrier medium 114 is water, and an example of a site is a site in muscle tissue or in skin tissue. It is also noted that eventually the fluorescent nanoparticles 116 will stop fluorescing and the carrier medium 114, including the fluorescent nanoparticles 116, will leave the injected patient tissue 118.

In one embodiment of the second method, the carrier medium 114, including the fluorescent nanoparticles 116, is injected inside tissue 118 of the patient 110 using a syringe 140. In one application, the predetermined shape is a substantially circular or spherical shape. In a first variation, the distal tip of the syringe 140 pierces the external skin of the patient. In one variation, an invisible ink outline of the predetermined shape is made on the skin surface (such as the ultraviolet-ink outline made visible under ultraviolet light) or a clear adhesive decal outline of the predetermined shape is placed on the skin surface. In one modification, an ink or visible dye is added to the carrier medium 114 to provide a surface landmark reference in-the external skin of the patient 110. In a second variation, not shown, the distal tip of the syringe extends from the distal end of an endoscope which is disposed inside a body lumen of the patient, wherein the distal tip of the syringe pierces the outer layer of a wall of the body lumen. Other variations, applications, and embodiments are left to the artisan.

It is noted that the implementations, enablements, employments, etc. of the first method are equally applicable to the second method. It is also noted that FIG. 4 shows a computer 134, additional image data 136, and a monitor 138.

Several benefits and advantages are obtained from one or more of the methods of the invention. In one example of the first method, the fluorescent nanoparticles in the image data act as a fiducial in creating an image representation of the image data, wherein the known shape of the specific tissue is more readily identified by the computer as the fiducial when fluorescent nanoparticles are used than when fluorescent nanoparticles are not used. In one example of the second method, the fluorescent nanoparticles in the image data act as a fiducial in creating an image representation of the image data, wherein the fluorescent nanoparticles in the predetermined shape of the injected carrier medium allow identification by the computer of the predetermined shape as the fiducial.

While the present invention has been illustrated by several methods, and enablements, applications, etc. thereof, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A method for medically imaging a patient comprising:
a) obtaining a carrier medium containing fluorescent nanoparticles;
b) disposing the carrier medium containing the fluorescent nanoparticles inside the patient at a site where the carrier medium will migrate to, and be picked up by, specific tissue of the patient, wherein the specific tissue has a known shape;
c) activating the fluorescent nanoparticles to fluoresce;
d) obtaining image data of the patient including the specific tissue after the specific tissue has picked-up the fluorescing fluorescent nanoparticles;
e) identifying the known shape of the specific tissue in the image data;
f) creating an image representation of the image data using the identified known shape as a fiducial; and
g) displaying an image of the image representation of the image data.

2. The method of claim 1, wherein the image data is magnetic resonance (MRI) image data.

3. The method of claim 1, wherein the image data is computerized tomography (CT) image data.

4. The method of claim 1, wherein the image data is positive electron emission (PET) images

5. The method of claim 1, wherein the image data is fluoroscope image data.

6. The method of claim 1, wherein steps e) and f) are performed by a computer.

7. The method of claim 6, wherein step e) is performed by the computer using a segmentation subroutine.

8. The method of claim 1, also including the steps of:
h) obtaining additional image data of the patient including the specific tissue;
i) creating an image representation of the additional image data; and
j) displaying an image of the image representation of the additional image data registered and overlaid with the displayed image of the image representation of the image data.

9. The method of claim 8, wherein the additional image data and the image data are obtained from different imaging modalities.

10. The method of claim 8, wherein the image data is pre-acquired image data and wherein the additional image data is real-time image data.

11. A method for medically imaging a patient comprising:
a) obtaining a carrier medium containing fluorescent nanoparticles;
b) injecting the carrier medium containing the fluorescent nanoparticles inside tissue of the patient at a site where the carrier medium will be localized in the injected tissue, wherein the injecting deposits the carrier medium in a predetermined shape in the injected tissue;
c) activating the fluorescent nanoparticles to fluoresce;
d) obtaining image data of the patient including the injected tissue;
e) identifying the predetermined shape of the injected carrier medium in the image data;
f) creating an image representation of the image data using the identified predetermined shape as a fiducial; and
g) displaying an image of the image representation of the image data.

12. The method of claim 1 1, wherein the image data is magnetic resonance (MRI) image data.

13. The method of claim 11, wherein the image data is computerized tomography (CT) image data.

14. The method of claim 11, wherein the image data is positive electron emission (PET) images

15. The method of claim 11, wherein the image data is fluoroscope image data.

16. The method of claim 11, wherein steps e) and f) are performed by a computer.

17. The method of claim 16, wherein step e) is performed by the computer using a segmentation subroutine.

18. The method of claim 11, also including the steps of:
h) obtaining additional image data of the patient including the specific tissue;
i) creating an image representation of the additional image data; and
j) displaying an image of the image representation of the additional image data registered and overlaid with the displayed image of the image representation of the image data.

19. The method of claim 18, wherein the additional image data and the image data are obtained from different imaging modalities.

20. The method of claim 18, wherein the image data is pre-acquired image data and wherein the additional image data is real-time image data.
